(19) United States
(12) Patent Application Publication
(10) Pub. No.: US 2018/0327292 A1
(43) Pub. Date: Nov. 15, 2018

(54) SYSTEM AND METHOD FOR WASTEWATER TREATMENT PROCESS CONTROL

(71) Applicant: SEMBCORP INDUSTRIES LTD, Singapore (SG)

(72) Inventors: Nan LI, Singapore (SG); Woh Peng Aaron YUEN, Singapore (SG); Wangdong NI, Singapore (SG); Zhengzhi ZHAO, Singapore (SG); Kwee Hock SIM, Singapore (SG)

(73) Assignee: SEMBCORP INDUSTRIES LTD, Singapore (SG)

(21) Appl. No.: 15/740,348

(22) PCT Filed: Apr. 18, 2017
(86) PCT No.: PCT/SG2017/050213

§ 371 Date: Dec. 28, 2017

(30) Foreign Application Priority Data
Apr. 18, 2016 (SG) PCT/SG2016/050180

(51) Int. Cl. C02F 3/00; C02F 3/28; C02F 3/30; G01N 33/18

(52) U.S. Cl. CPC C02F 3/006; C02F 3/2846; C02F 3/30; G05B 13/048; C02F 2203/002; C02F 2209/006; C02F 2209/20; G01N 33/1846

(57) ABSTRACT

A system for wastewater treatment process control comprising a set of measuring means arranged to obtain a dataset, the dataset comprises a plurality of process variables related to a parameter of the wastewater treatment process; a prediction module arranged to receive the dataset and predict the parameter of wastewater treatment process based on a soft sensor; a troubleshooting module arranged to compare the predicted parameter with a predetermined criterion; wherein if the predicted parameter does not satisfy the predetermined criterion; the troubleshooting module is operable to identify at least one process variable from the plurality of process variables which causes the predicted parameter not to satisfy the predetermined criterion and determine whether the identified at least one process variable from the plurality of process variables is controllable. An optimisation module for use in a wastewater treatment system is also disclosed.

### FIELD OF INVENTION

The present invention relates to a system and method for process control in wastewater treatment. In particular, this system and method is suitable for, but not limited to control a parameter of an Expanded Granular Sludge Bed (EGSB) process and will be described in such context.

### BACKGROUND ART

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Throughout the specification, unless the context requires otherwise, the word “comprise” or variations such as “comprises” or “comprising”, will be understood to imply the inclusion of a stated integer or group of integers but not the exclusion of any other integer or group of integers.

Furthermore, throughout the specification, unless the context requires otherwise, the word “include” or variations such as “includes” or “including”, will be understood to imply the inclusion of a stated integer or group of integers but not the exclusion of any other integer or group of integers.

Biological wastewater treatment processes are widely used and typically comprise anaerobic wastewater treatment and aerobic wastewater treatment.

In aerobic wastewater treatment processes, microorganisms such as bacteria, protozoa and fungi use dissolved oxygen as a key component to carry out carbonaceous Biological Oxygen Demand (BOD) degradation and removal of Ammonia waste via nitrification. When the process is well operated, aerobic wastewater treatment processes are robust and reliable in treating wastewater to the required quality for effluent discharge. Examples of aerobic wastewater processes commonly applied for industrial wastewater treatment include activated sludge processes, membrane bioreactors (suspended growth systems) or trickling filter and carrier-based treatment system such as the Moving Bed Biofilm Reactor (attached growth systems). In each of the above processes, oxygen is supplied to the microorganisms in the form of air delivered by rotating equipment such as air blowers and compressors. The dissolved oxygen in the wastewater is utilized by the microorganisms as electron acceptors for aerobic decomposition of the carbonaceous BOD. At the end of the aerobic wastewater treatment process, BOD and other contaminants in the wastewater are oxidized to carbon dioxide and additional biomass.

Anaerobic wastewater treatment processes are industrial processes that use microorganisms to breakdown contaminants in the wastewater under oxygen free environments. Anaerobic wastewater treatment has developed to be a prominent alternative to conventional aerobic treatment due to advantages such as reduced sludge production, low energy consumption and ability for energy recovery via methane generated from the process. Anaerobic wastewater treatment processes may also be operated at higher rates (organic loading and volumetric) as it is not limited by oxygen availability. Typical variants of anaerobic wastewater processes used in the industry include anaerobic filters, down flow stationary fixed-film reactors, fluidized bed anaerobic reactors and Upflow Anaerobic Sludge Blanket Reactors (UASB). The Expanded Granular Sludge Bed (EGSB) reactor is a specific application of the USAB with high wastewater treatment rate. In the EGSB process, wastewater passes through the reactor with high upward-flow velocity.

In both aerobic and anaerobic wastewater treatment processes, achieving a stable and economical operation remains a complex challenge for plant operators. Such complexity can be attributed to the high sensitivity nature of the processes to various factors such as organic loading disturbance, dynamic changes in treatment conditions and composition of various substances in the influent wastewater. To illustrate by way of an example, the biological degradation of contaminants in the biological wastewater treatment proceeds as a series of biological reactions. Each of these sequential biological reactions involves distinct species of microorganisms. As such, load and composition changes of the influent wastewater can upset the delicate equilibrium that exists between the various species of microorganisms present in the system. Similarly, determination of optimum influent characteristics for operating the biological treatment process is highly complex because the process is highly sensitive to the presence of various toxic pollutants that may affect any of the microorganism species present. The biomass generation rate will further complicate its control as microorganism that is lost unintentionally through hydraulic washout or toxic inhibition (during process upsets) requires extended durations for the lost biomass to be replaced.

Existing methods, including mathematical models such as soft sensors have been deployed in the control and forecast of wastewater treatment control. Despite these existing methods in forecasting wastewater treatment control, there still exists a need for an advanced monitoring and process control strategy that allows early detection and isolation of faults that may lead to catastrophic failure of the wastewater treatment systems would immensely benefit wastewater treatment process. Further, the ability for an operator to provide early intervention through implementation of corrective instructions would prevent minor process deviations from developing into serious operational problems.

It is an object of the invention to meet the above need at least in part.

### SUMMARY OF THE INVENTION

In accordance with a first aspect of the invention there is a system for wastewater treatment process control comprising a plurality of measuring means arranged to obtain a dataset, the dataset comprises a plurality of process variables related to a parameter of the wastewater treatment process; a prediction module arranged to receive the dataset and predict the parameter of wastewater treatment process utilizing a mathematical model, the mathematical model arranged to obtain the dataset as input and provides a predicted parameter as an output; a troubleshooting module arranged to compare the predicted parameter with a predetermined criterion; wherein if the predicted parameter does not satisfy the predetermined criterion; the troubleshooting module is operable to identify at least one process variable from the plurality of process variables which causes the predicted parameter not to satisfy the predetermined criterion.

The parameter of the wastewater treatment process may be an effluent parameter of the wastewater treatment process.

The mathematical model for prediction may be a soft sensor. In some embodiments, the mathematical model comprises a moving-window partial least squares regression algorithm.

The identification of the at least one process variable which does not satisfy the predetermined criterion may be facilitated based on a Hotelling T² or Q/SPE statistic.

The troubleshooting module may be operable to determine whether the identified at least one process variable from the plurality of process variables is controllable. In some embodiments, if the at least one process variable is controllable, the troubleshooting module then proceeds to obtain the median value of the at least one controllable process variable and determines if the at least one controllable process variable is a root cause.

The determination of whether the at least one controllable process variable is a root cause may include comparing the median value of the at least one controllable process variable against a range of values the at least one controllable process variable operates under normal condition. As an example, if the controllable process variable falls outside the range of values, the controllable process variable is classified as a root cause. The root cause may be further classified as either a qualitative or a quantitative root cause.

If the root cause is a quantitative root cause, further calculations are provided or performed to calculate an adjustment to the at least one process variable.

In some embodiments, the troubleshooting module is arranged in data communication with a database, and operable to access the database to retrieve at least one corrective instruction to adjust the controllable process variable based on a set of pre-defined rules. Such pre-defined rules may be maintained in a recommendation database.

In some embodiments, the system further comprises a prognosis module operable to simulate the impact of the adjustment of the controllable process variable on the parameter or predicted parameter of the wastewater treatment process. The prognosis module may be arranged in data communication with the troubleshooting module to receive the controllable process variable after adjustment in accordance with the corrective instruction.

Alternatively, the prognosis module may bypass the troubleshooting module and arranged directly in data communication with the prediction module. In such an arrangement, the prognosis module is operable to simulate the impact of an adjustment of at least one process variable on the parameter of the wastewater treatment process.

In some embodiments, an optimization module may be arranged in data communication with the prediction module, troubleshooting module or prognosis module to optimize the plurality of process variables and parameter of wastewater treatment process with respect to at least one objective function.

In accordance with a second aspect of the invention there is a method for wastewater treatment process control comprising the steps of: obtaining from a plurality of measuring means a dataset, the dataset comprises a plurality of process variables related to a parameter of the wastewater treatment process; receiving the dataset as input at a prediction module and predicting the parameter of wastewater treatment process based on a mathematical model; comparing the predicted parameter with a predetermined criterion; wherein if the predicted parameter does not satisfy the predetermined criterion; the troubleshooting module is operable to identify at least one process variable from the plurality of process variables which causes the predicted parameter not to satisfy the predetermined criterion.

In accordance with a third aspect of the invention there is a troubleshooting module for use in wastewater treatment process control, comprising at least one processor in data communication with a plurality of measuring means to receive a dataset, the dataset comprises a plurality of process variables related to a parameter of the wastewater treatment process, and a value of the parameter of the wastewater treatment process; and thereafter compare the value of the parameter with a predetermined criterion; wherein if the parameter does not satisfy the predetermined criterion; the troubleshooting module is operable to identify at least one process variable from the plurality of process variables which causes the predicted parameter not to satisfy the predetermined criterion.

The troubleshooting module may be arranged in data communication with a historical database and/or a recommendation database to retrieve information relating to the normal operating range (normal range) of each process variable and the parameter of the wastewater treatment process.

In some embodiments, identification of the at least one process variable from the plurality of process variables is facilitated based on a Hotelling T² or Q/SPE statistic.

The troubleshooting module may be operable to determine whether the identified at least one process variable is controllable. A controllable process variable refers to an identified process variable that is able to be adjusted or controlled. Conversely, an uncontrollable abnormal process variable refers to an abnormal variable that is unable to be adjusted or controlled.

If the at least one process variable is controllable, the median value of the at least one controllable process variable is obtained and the troubleshooting module is operable to determine if the at least one controllable process variable is a root cause.

The determination of whether the at least one controllable process variable is a root cause may include comparing the median value of the at least one controllable process variable against a normal range. In some embodiments, if the at least one controllable process variable falls outside the normal range, the controllable process variable is classified as a root cause. The root cause may be further classified as either a qualitative or a quantitative root cause. If the root cause is classified as a quantitative root cause, an adjustment to the process variable is calculated. The adjustment may be facilitated by a recommendation database for retrieval of at least one corrective instruction to adjust the controllable process variable. Such corrective instruction may be based on pre-defined rules.

The troubleshooting module may be arranged in data communication with a prognosis module, the prognosis module operable to simulate the impact of the adjustment of the controllable process variable on the parameter of the wastewater treatment process.

The prognosis module may further be operable to simulate the impact of adjustments of any other process variable(s) on the parameter of the wastewater treatment process.

In some embodiments, the troubleshooting module may further comprise a list of a plurality of quantitative and/or qualitative root causes and corresponding correction actions, wherein each corresponding adjustment is a corrective action.

In some embodiments, the troubleshooting module may comprise an optimization module arranged to permute the corrective actions in various combinations in accordance with the formula

$${\sum }_{r = 1}^{n} C ( n , r )$$

where n denotes the total number of corrective actions identified. A simulated parameter corresponding to each corrective action may then be determined and each simulated parameter is compared with the predetermined criterion.

In a scenario, if the simulated parameter satisfies the predetermined criterion, an objective function value is calculated, and where there comprises a plurality of objective function values, the lowest or highest objective function value is selected as an optimal solution. If no simulated parameter is found to satisfy the predetermined criterion, a next best alternative based on adjusting a predetermined process variable is selected as the optimal solution.

In accordance with a fourth aspect of the invention there is an optimization module for use in wastewater treatment process control, comprising at least one processor in data communication with a plurality of measuring means to receive a dataset, the dataset comprises a plurality of process variables related to a parameter of the wastewater treatment process; wherein the at least one processor is operable to optimize the parameter and the plurality of process variables with respect to an objective function, and thereafter a corrective instruction is determined based on the optimization.

A possible objective function is an overall process operating cost, which is to be minimized. The overall process operating cost may be further optimized with respect to a cost related to implementing the corrective instruction.

In some embodiments, the optimization module is utilized for use in wastewater treatment system, the wastewater treatment system comprises at least one anaerobic sub-system and at least one aerobic sub-system, the optimization module comprises at least one processor arranged in data communication with a plurality of measuring means to receive a dataset, the dataset comprises a plurality of process variables related to a parameter of the wastewater treatment process;

wherein the at least one processor is operable to optimize the parameter and the plurality of process variables with respect to an objective function, and wherein the objective function comprises minimization of an overall operating expense of the wastewater treatment system.

The minimization of the overall operating expense may include minimizing the effluent TOC concentration in the anaerobic sub-system, and/or maximizing the effluent TOC concentration in the aerobic sub-system.

### BRIEF DESCRIPTION OF THE DRAWINGS

The present invention will now be described, by way of example only, with reference to the accompanying drawings, in which:

FIG. 1 is a schematic diagram of a wastewater treatment process plant in the form of an Expanded Granular Sludge Bed Reactor system.

FIG. 2 is a flow chart illustrating one embodiment of the invention.

FIG. 3 is a block diagram of a system for wastewater treatment process control in accordance with some embodiments of the invention;

FIG. 4 shows the result of predicting an effluent parameter (TOC) of the WWTP.

FIG. 5 is a graphical user interface to allow a user to enter the TOC criteria to determine when the troubleshooting module is activated prior to making prediction of the EGSB performance.

FIG. 6 is a flow-chart showing the work flow in the troubleshooting module.

FIG. 7 shows the results of a typical statistical analysis performed by the Troubleshooting module based on Hotelling T² or Q/SPE Statistical Models.

FIGS. 8a and 8b illustrate a pseudocode for performing a process calculation when triggered.

FIG. 9 shows the simulated effect of adopting the corrective actions generated by the Troubleshooting module.

FIG. 10 shows the process parameters and their values to allow the user to make changes to get the projected TOC in the Prognosis Module.

FIG. 11 shows the projected TOC from prognosis module by changing T-01 wastewater TOC (lab) from 825.3 mg/L to 700 mg/L.

FIG. 12 shows the optimized effluent parameter (the example here is TOO value) and the corresponding optimized parameters.

FIG. 13 shows a list of recommendations/corrective actions to be implemented to achieve optimized result for the TOC.

FIG. 14 shows a wastewater treatment system that comprises an anaerobic and an aerobic treatment sub-systems in accordance with some embodiments of the invention, wherein the optimization module is utilized to minimize an overall operating expense (OPEX) of the system.

FIG. 15 shows a method for optimizing OPEX based on an identified list of root causes.

FIG. 16 shows exemplary applications of the method of FIG. 15 in accordance with some embodiments of the invention.

### DESCRIPTION OF EMBODIMENTS OF THE INVENTION

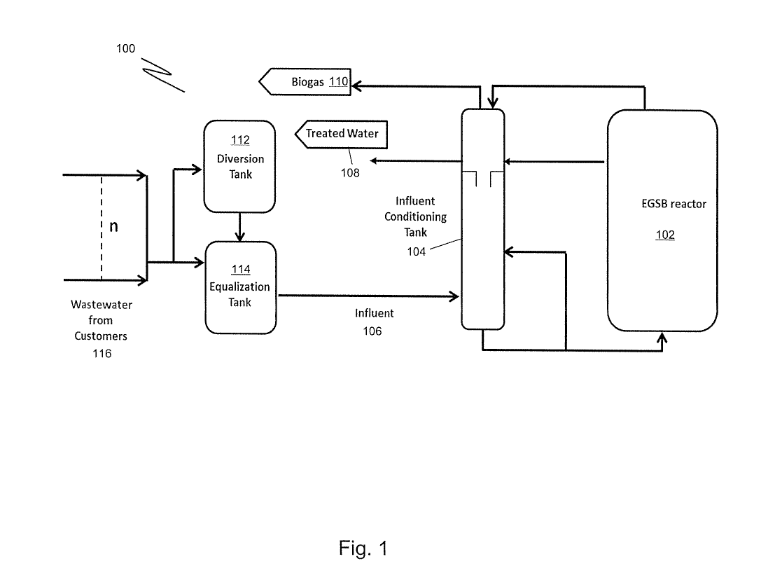

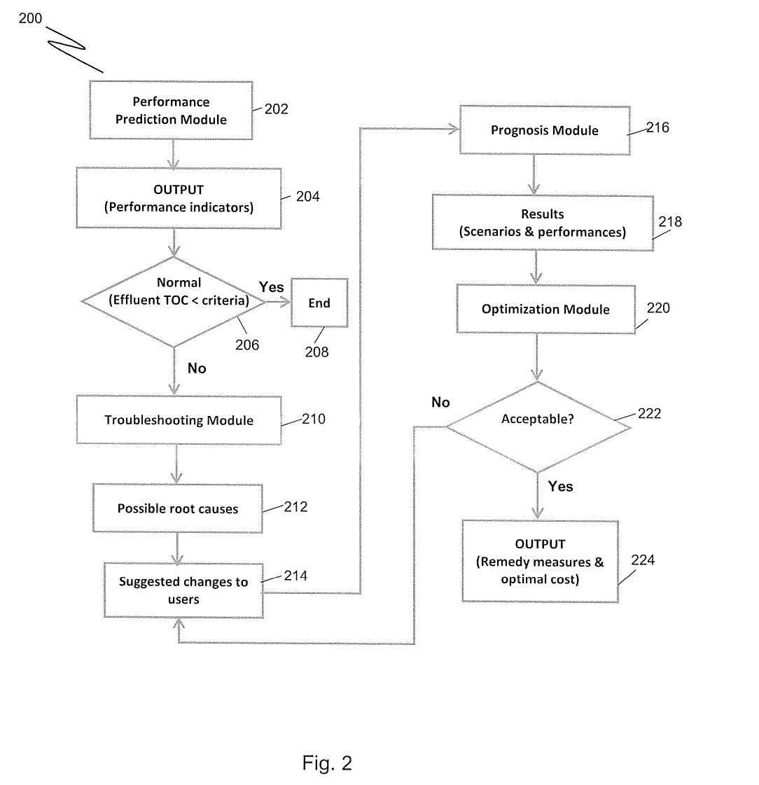

In accordance with an embodiment of the invention and as shown in FIG. 2, there is a method 200 for wastewater treatment process (WWTP) control in a wastewater treatment process system, such as the EGSB system 100. With reference to FIG. 1, a typical EGSB system 100 comprises an EGSB reactor 102, an influent conditioning tank 104, a diversion tank 112 and an equalization tank 114. Influent wastewater from different sources or customers 116 may be fed into either the diversion tank 112 or the equalization tank 114. The main purpose of the equalization tank 114 is to dampen fluctuations in flow rate, temperature and contaminant concentrations of the influent wastewater so as to maintain the operational performance of downstream processes. In the event when there is a surge in a parameter (pressure, flowrate, contaminant concentrations, etc.) of the wastewater from customers 116, the wastewater flow 116 may be diverted to the diversion tank 112 as a means of absorbing sudden change in the parameter of the wastewater from customers 116. Eventually, the influent wastewater from customers 116 is directed to the EGSB reactor 102 for treatment.

In the EGSB reactor 102, granulated microorganisms are contacted against influent wastewater from the influent conditioning tank 104 flowing at relatively high flow rates. After the wastewater is adequately treated in the EGSB reactor 102, it is discharged as treated water 108 (effluent). The consortium of microorganisms in the EGSB process breaks down the organic contaminants in the wastewater to form additional biomass (microorganisms), treated water 108 and energy in form of biogas 110, which may include methane (CH₄) and carbon dioxide (CO₂).

In order to monitor the various process variables and effluent parameters, such as for example Total Organic Carbon (TOC), the EGSB system comprises a plurality of measurement means located or positioned at a suitable location, i.e. around/within the EGSB system to obtain regular measurements of the process variables and/or process parameters, and effluent parameters. The measuring means may be one or more devices in the form of one or more physical sensors positioned in strategic locations in and/or around the EGSB plant system to obtain the measurements. In addition or in the alternative, samples of waste water may be obtained from the EGSB plant and measured in a laboratory environment to derive the process variables as known to a skilled person. These measurements obtained in a laboratory environment are referred to as laboratory measurements. Overtime, a historical database 30 comprising the measured process variables/parameters, and effluent parameters is built and regularly updated. The historical database 30 may be queried to obtain statistical calculations for each process variable and effluent parameter. The statistical calculations include mean, median, standard deviation, maximum, minimum values associated with each process variable and/or process parameter. For each process variable and/or parameter, a range of values under normal operating conditions (hereinafter referred to as ‘normal values’) may be derived from the data within the historical database 30.

In the context of the description, the term ‘parameter’ may be construed broadly to include constant, variables, calculated or derived values.

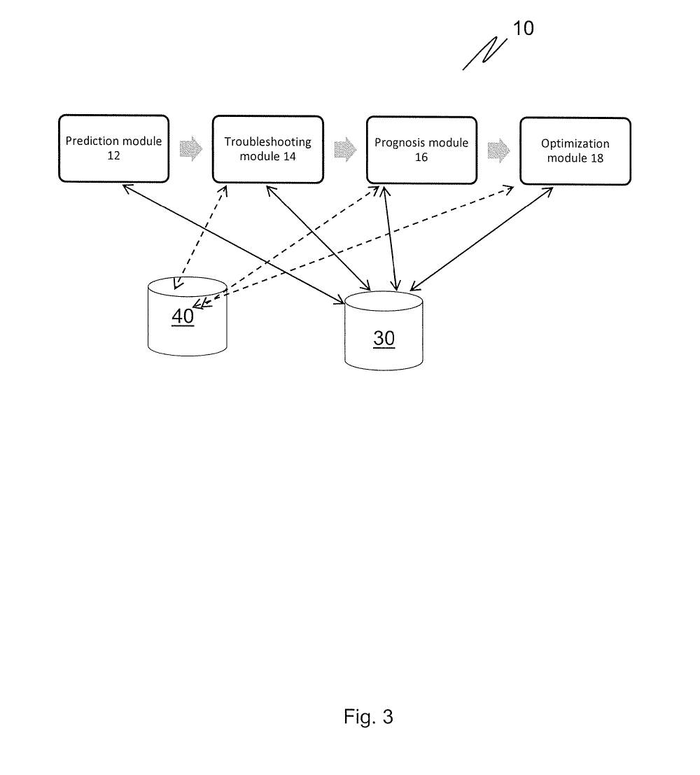

Referring to FIG. 3, there is a system 10 for wastewater treatment process control comprising a prediction module 12 arranged to receive from the plurality of measurement means a dataset, and predict an effluent parameter of the wastewater treatment process based on a first function. The first function may be in the form of a heuristic and/or statistical method, and may include supervised or unsupervised learning methods, which include the use of data within the historical database 30 as a ‘teacher’. The first function may be a mathematical model or a soft sensor. The first function is utilized to obtain at least the dataset as input and provides the predicted effluent parameter as an output.

The system 10 further comprises a troubleshooting module 14 arranged to compare the predicted parameter or an attribute of the predicted parameter with a predetermined criterion or criteria (elaborated below); wherein if the predicted parameter or the attribute does not satisfy the predetermined criterion; the troubleshooting module is operable to identify at least one process variable from the plurality of process variables that may have caused the predicted parameter to not satisfy the user defined criterion.

The system may further comprise a prognosis module 16 and an optimization module 18. Each of the prediction module 12, the troubleshooting module 14, the prognosis module 16 and the optimization module 18 may comprise one or more computer processors in the form of servers, arranged in a distributed or integrated arrangement. The processors and/or servers are arranged in data communication to obtain the dataset from the plurality of measuring means. The processors and/or servers may also be arranged in data communication with the historical database 30 and a recommendation database 40. Further, the prediction module 12, the troubleshooting module 14, the prognosis module 16 and the optimization module 18 may be arranged in various permutations with respect to one another.

In some embodiments, the method 200 for wastewater treatment process control may be implemented as software codes on one or more non-transitory computer readable medium within the plurality of servers.

Referring again to FIG. 2, the method 200 to control the WWTP may comprise steps as performed by the prediction module 12, also used interchangeably with the term Performance Prediction Module. In step 202, the Performance Prediction Module 12 obtains a dataset comprising process parameters or variables related to the EGSB system. The dataset may comprise:

a. real time data obtained from the plurality of measuring means;

b. laboratory measurements;

c. data stored in the historical database 30; and/or

d. a combination of real time data, laboratory measurements and data stored in the historical database 30.

After obtaining the dataset, the Performance Prediction Module 12 may utilize the first function, which can be heuristic or statistical method or function, to predict the effluent TOC for one or more EGSB reactor (step 202). In various embodiments, the first function may be, but not limited to, factor-based models like Principal Components Analysis, Partial Least Squares or Principal Components Regression models. In other embodiments, non-factor-based models such as, but not limited to, Multiple Linear Regression, Artificial Neural Networks or Support Vector Machine may be used. In the preferred embodiment, the first function may be in the form of a Moving Window Partial Least Squares algorithm, which may be in the form of equation (1) below.

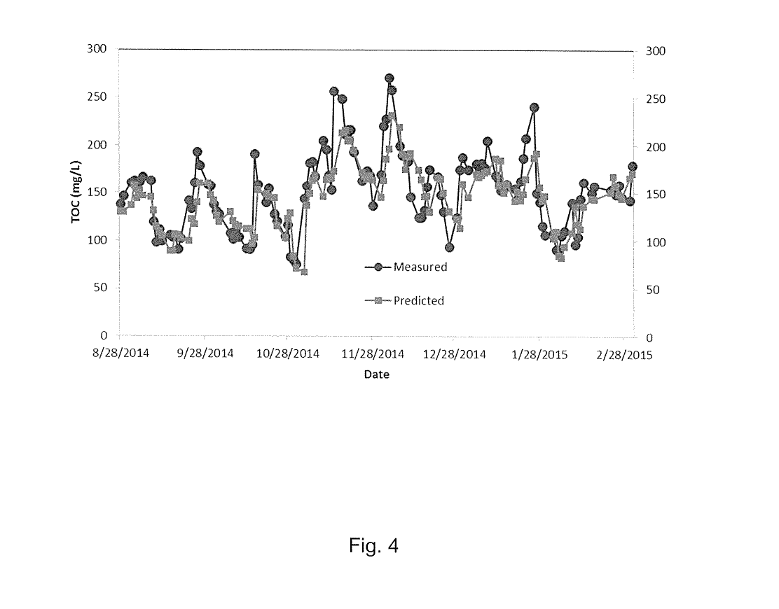

With reference to FIG. 4, a comparison between the predicted TOC for an EGSB reactor (see square symbols in FIG. 4) and the measured TOC (see circle symbols in FIG. 4) of the wastewater, as obtained from the historical database, using a Moving Window Partial Least Squares method (MWPLS) is shown. It may be observed that the MWPLS method is capable of accurately predicting the effluent parameter (TOC) using process parameters that are easily accessible instead of relying on on-site analysers or lab measurements. Others parameters that are not easily measured on site includes Biochemical Oxygen Demand (BOD), ortho-phosphates (PO4-P) and (ammonium nitrogen) NH4-N.

MWPLS is advantageous as the use of the moving-window strategy allows the model to self-update and to incorporate input parameters that are aligned with the plant's latest status, at the same time, removing system information that is outdated and non-informative.

By way of an example, MWPLS makes use of a fixed window (where the window size n is usually equal to the number of samples or the number of days in the historical data set) with an adaptation strategy which alternatively updates the window in a sample-wise manner and recalculates the PLS model over the updated window. If the historical data set X=(x₁, . . . , xₙ)^{T} and Y=(y₁, . . . , yₙ)^{T} at time t−1, the window used in MWPLS with the newly obtained data pair xₜ and yₜ can be mathematically expressed as equation (1) as follows:

$${X}_{t} = [ \begin{matrix}{x}_{2} \\ … \\ {x}_{n} \\ {x}_{t}\end{matrix} ] {Y}_{t} = [ \begin{matrix}{y}_{2} \\ … \\ {y}_{n} \\ {y}_{t}\end{matrix} ] \qquad ( 1 )$$

The fixed window is moved sample by sample to track process dynamics. The vector Xₜ may be formed comprising a set of input process variables and/or process parameters. The vector Yₜ may be formed comprising a set of output effluent parameter, in this case TOC, to be predicted.

The input process variables or parameters include, but are not limited to the following real time measurements and/or laboratory measurements related to the equalization tank 114):

a. wastewater level in tank (also referred to as tank level);

b. outlet temperature;

c. wastewater pH;

d. waste water TOC;

e. inlet temperature;

f. total suspended solid in wastewater;

g. Volatile Fatty Acid in wastewater;

h. wastewater alkalinity (may be a subset of pH).

The input process variables or parameters may include the following real time measurements and/or laboratory measurements related to the influent conditioning tank 104:

a. flowrate of wastewater (from Equalization Tank 114 to Conditioning Tank 104);

b. pH of wastewater in at least one conditioning tank 104;

c. control valve (including but not limited to feed flow valve) opening timing/frequency or state to control flowrate of wastewater to conditioning tank 104;

d. valve opening timing/frequency or state for flow control between conditioning tank 104 and EGSB reactor 102;

The input process variables or parameters may include the following real time measurements and/or laboratory measurements related to the biogas pressure; temperature, flowrates of wastewater flowing pass heat exchangers etc.

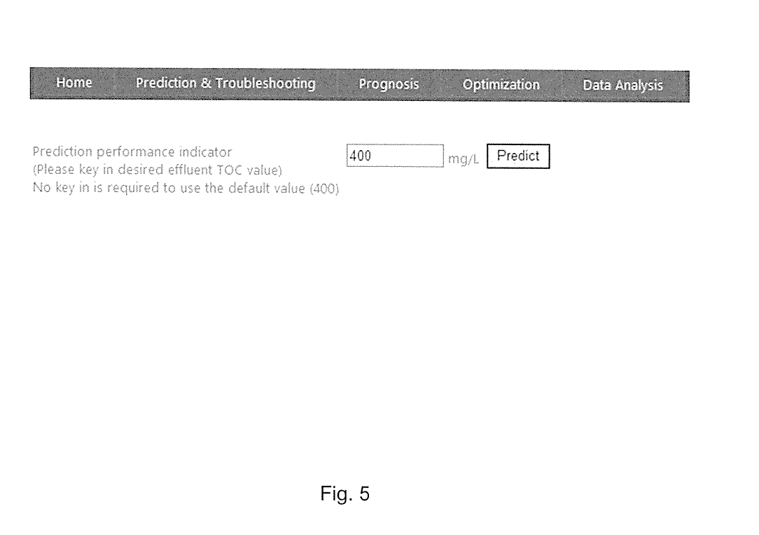

After predicting the effluent TOC in step 204, the prediction module 12 may further compare the predicted effluent TOC value of the corresponding EGSB reactor 102 against the user defined TOC value in step 206. The user defined TOC value may be inputted by the user via the graphic user interface as shown in FIG. 5. If the predicted effluent TOC satisfies one or more predetermined criteria, nothing is done (step 208).

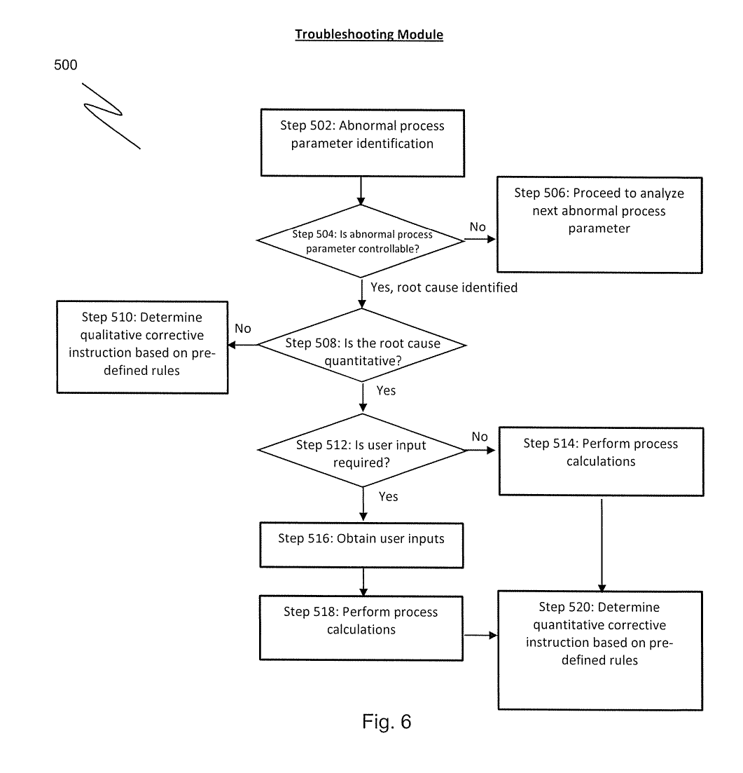

In the event that the predicted effluent TOC does not satisfy one or more user predetermined user defined criterion/criteria, the troubleshooting module 14 is triggered in step 210. FIG. 6 shows a more detailed work flow 500 of the troubleshooting module 14 carried out in the method steps 212 and 214. In this case, the predetermined user defined criterion/criteria is in the form of a maximum value allowable for the predicted effluent TOC. More generally, the predetermined user defined criterion/criteria for an effluent or process parameter may either be in the form of a maximum value allowable, a range of acceptable or allowable values, or a minimum value allowable.

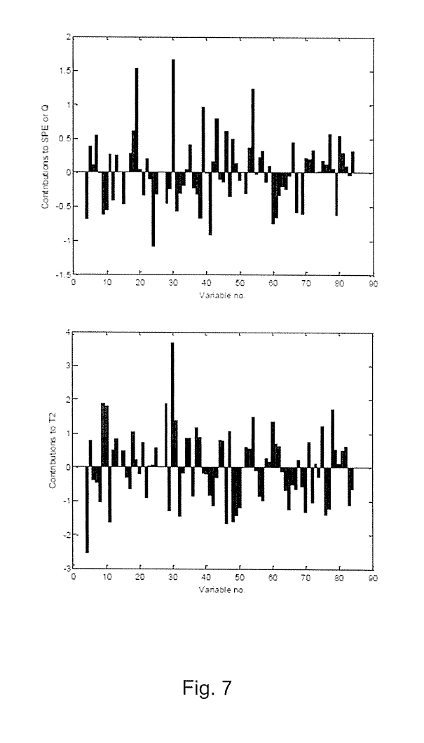

In step 502, upon detection that the predicted effluent TOC does not satisfy the predetermined criterion/criteria, the troubleshooting module 14 is operable to identify at least one, but typically a plurality of possible process variables or parameters (hereinafter referred to as abnormal process parameters). By way of an example the abnormal process parameters may comprise ten process variables/parameters, which may have caused the predicted effluent TOC to not meet the predetermined criterion/criteria. Using the example of the predetermined criterion in the form of a range of acceptable or maximum value allowable for the predicted effluent TOC, any TOC value out of range may trigger the activation of the troubleshooting module 14. In some embodiments, the Troubleshooting module 14 may utilize, in step 502, multivariate statistical analysis (such as, but not limited to, Hotelling T² or Q/SPE statistics) to identify the plurality of abnormal process parameters. It may be appreciated that the weightage or contribution of the multivariate statistical analysis is useful to identify the process variables or parameters which contribute most to the predicted TOO not satisfying the predetermined criterion. Hence, the process parameters associated with the highest weightage or contribution as illustrated in FIG. 7 may be considered or determined as the abnormal process parameters. In other embodiments, the identification of abnormal parameters may be performed using statistical process control (SPC), and operation and/or expert process knowledge.

In step 504, the troubleshooting module 14 may further classify the identified abnormal process variables to those which are controllable and then compare the median value of each controllable abnormal process parameter against its normal range, the normal range obtainable from the historical database 30. A controllable abnormal process variable refers to an abnormal variable that is able to be adjusted or controlled. An uncontrollable abnormal process variable refers to an abnormal variable that is unable to be adjusted or controlled. Controllable abnormal process parameters with median value falling out of the normal range are classified as a “root cause”. It is appreciated that there may be more than one root causes associated with different process variables.

Upon identifying the root causes, a corrective action may then be determined from the recommendation database 40. The recommendation database 40 contains a compilation of at least one potential corrective instruction corresponding to each specific root cause. It may be appreciated that user experiences in wastewater treatment process control over time provide a wealth of information, allowing users or wastewater treatment plant operators to understand the status of the process and assisting them to make appropriate actions to remove abnormalities resulting from the process and minimize unnecessary operational cost resulting from process upsets. Such user experiences may be captured at least as part of the recommendation database 40 comprising a list of potential corrective instruction corresponding to each specific root cause.

In various embodiments, the root causes may be broadly categorized into either “Quantitative” or “Qualitative”. Quantitative root causes are those which have corresponding potential corrective instructions which involve process calculations and qualitative root causes are those which have corresponding potential corrective instructions which do not involve process calculations. In some embodiments, process calculation involves calculating a process-related variable/parameter which may be expressed as a function of a process parameter and/or process-related user input. The process-related variable/parameter may also be expressed as a function of a process parameter with/without process-related user input.

When a qualitative root cause is identified in step 508, the appropriate corrective instruction may then be looked-up or determined from a corresponding list of potential corrective instructions using pre-defined rules in the recommendation database 40. It is to be appreciated that in various embodiments, the corresponding list of potential corrective instructions may contain at least two potential corrective instructions. Hence, Boolean logic may be used to determine the more applicable corrective instruction from the two potential corrective instructions. The Boolean operators used may include the “more than” and “less than” operators.

For example, when abnormal process variable pH of equalization tank 01 (“T-01”), i.e. “T-01 pH” is identified as a root cause, the Boolean operator compares:

1) “T-01 pH” with the minimum value of 4 using the “less than operator”, and

2) “T-01 pH” with the maximum value of 9 using the “more than operator”.

As only one of the Boolean operators will return “TRUE” as the Boolean result, the recommendation database 40 will return the recommended corrective instruction corresponding to the “TRUE” result. If the current “T-01 pH” is 3, then the recommendation database 40 will return the corrective instruction as “The wastewater entering T-01 is too acidic. Check pH of incoming wastewater streams for low pH waste entering the tank. Monitor the level of caustic in T-400”. In this case, T-400 is the caustic storage tank and it is important that the operator monitors the amount (level) of caustic in this tank in situations whereby the incoming wastewater is acidic so as to ensure there is sufficient caustic for pH adjustment.

It is to be appreciated that the qualitative corrective instructions generated in step 510 extend beyond merely providing a warning that the abnormal process parameter is out of normal range. It gives a short context/synopsis of the existing problem “T-01 is too acidic” and provides plant operators with instructions to solve the existing problem. In addition, the qualitative corrective instructions may include reminders to the operators to monitor the pH and ensure that there is sufficient chemical inventory for process adjustment” or similar (“Check pH of incoming wastewater streams for low pH waste entering the tank. Monitor the level of caustic in T-400”).

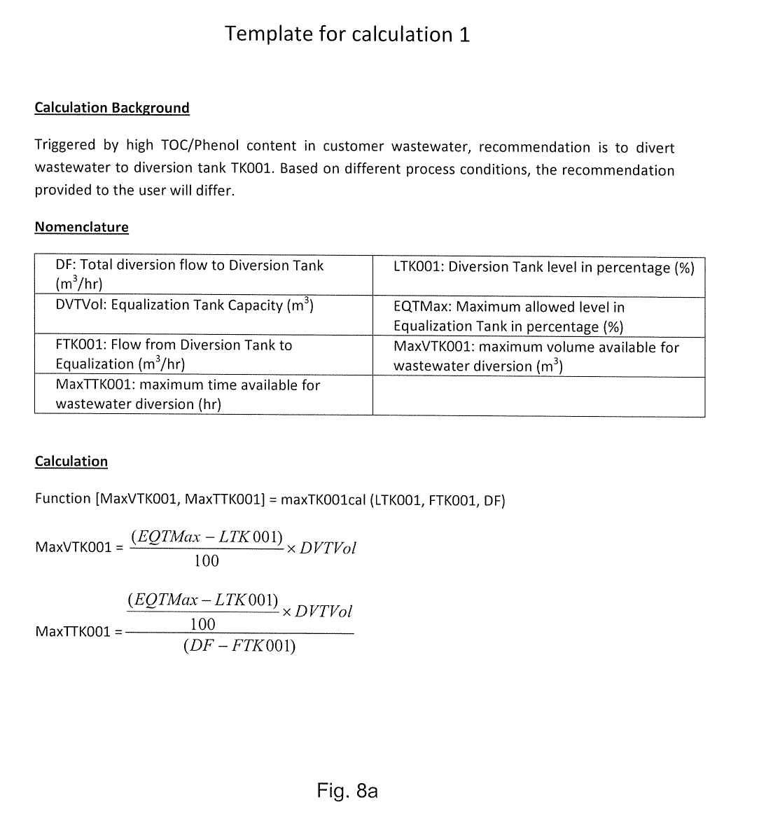

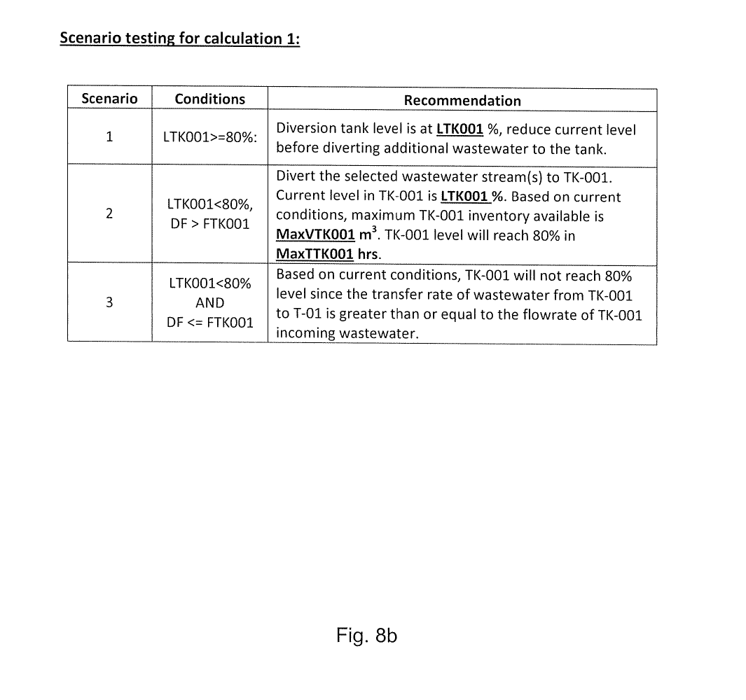

When a quantitative root cause is identified in step 508, the troubleshooting module 14 is operable to determine if process-related user inputs are required in step 512 before performing process calculations to determine a process-related parameter in step 514 (without user inputs) or step 518 (with user inputs). In various embodiments, process-related user inputs are necessary to allow for some user discretion and flexibility in the WWTP process control. The process calculations performed in either steps 514 or 518 are based on at least the process-related user inputs and/or existing process parameters. Following the process calculations, appropriate corrective action may then be determined in step 520 from the recommendation database 40 based on at least the calculated process-related parameter and pre-defined rules or logic. For example, when the abnormal controllable process parameter is identified to be “Customer A”, the processor or recommendation database 40 proceeds to perform “Calculation 1” to determine the corrective instruction. FIG. 8 is a pseudocode for “Calculation 1” showing the calculation of the maximum volume available for wastewater diversion in metres cube (m³) “MaxVTK001” and maximum time available for wastewater diversion in hours (hr) “MaxTTK001” process-related parameters which may be expressed as a function of existing process parameters and process-related user inputs such as:

(i.) DF: Total diversion flow to Diversion Tank (in cubic metres (m³) per hour)

(ii.) DVTVol: Equalization Tank Capacity (in m³)

(iii.) FTK001: Flow from Diversion Tank to Equalization tank (in m³ per hour)

(iv.) LTK001: Diversion Tank level in percentage filled (%)

(v.) EQTMax: Maximum allowed level in Equalization Tank in percentage filled (%)

Following the calculation of the process-related parameters “MaxVTK001” and “MaxTTK001”, Boolean logic may then be used to determine the corrective instruction from a list of possible corrective instructions based on a set of pre-defined rules in step 520. In various embodiments, there may be a need to use a combination of Boolean operators to determine the correction action due to the complexity of the WWTP. In this respect, the Boolean operators may be a combination of, but not limited to, “more than”, “less than”, “AND”, “NAND”, “OR” and “NOR” operators.

One advantage associated with having a list of corresponding possible corrective instructions for each quantitative root cause is to provide a secondary system of checks and balances so as to ensure that the WWTP which is already functioning in an abnormal state do not spiral out of control.

Referring back to “Calculation 1” as an example, the recommendation database 40 is queried to check the process parameter “LTK001” (Diversion Tank Level) first before determining the appropriate corrective instruction from the list of possible corrective instructions. In situations when there is high TOC/Phenol content in the customer's wastewater, one possible solution would be to divert the wastewater to the diversion tank. However, diverting the wastewater to the diversion tank without first checking the diversion tank level may result in overflowing especially if the diversion tank is near its maximum allowable capacity as in scenario 1 of “Calculation 1”. Consequently, diversion of wastewater will not have any corrective impact on the overall situation of the WWTP. Instead, overflowing of the diversion tank may lead to other safety and environmental concerns. Therefore, taking such an action may not only reduce the performance of the diversion tank but also degrades the overall performance of the EGSB system.

Even when the diversion tank has sufficient capacity, there is a need to prevent the aforementioned situation from happening if it is determined by Boolean logic that the diversion flow rate (DF) is greater than the diversion tank to equalization tank flow rate FTK001 (“DF>FTK001”). As illustrated in scenario 2 of “Calculation 1”, the process calculations serves to provide this safety net by determining:

1) the available capacity of the diversion tank at a particular juncture, and

2) the time it takes for the diversion tank to reach its maximum allowable capacity based on the diversion flowrate as chosen by the user.

Consequently, these quantitative information allows the user or plant operator to implement the corrective instruction informatively.

In scenario 3 of “Calculation 1”, if it is determined by Boolean logic that the diversion flow rate is less than the diversion tank to equalization tank flow rate (“DF>FTK001”) “AND” the diversion tank level is below 80% of its maximum allowable capacity (LTK001<80%), the user is able to perform wastewater diversion without any additional concerns. In various embodiments, it is to be appreciated that only one of the scenarios will return “TRUE” to ensure that the conflicting corrective instructions are not provided to the user. However, depending on the nature of the problem, there may also be scenarios (not explicitly shown) where there may be more than one “TRUE” Boolean result whereby the corrective instruction is a combination of at least two possible corrective instructions.

Although the Troubleshooting module 14 utilizes the recommendation database 40 which is a compilation of corrective actions based on the experiences of at least one domain expert or human operator, it achieves technical advantages that extend beyond mere automation of a task usually performed by a human.

For example, the corrective action issued to the operator is determined based on process condition of the wastewater plant at the point of abnormality and thus provides a “most relevant” solution to the issue. Also, having quantitative recommendations gives the plant operator clear, operable instructions. This gives the operator a clear picture of what needs to be done and the extent of the corrective actions and gives the operator an expectation of the extent of the corrective action required.

Further, the rule-based or logic-based recommendation database 40 eradicates subjectivity clouded judgements or judgements due to insufficient facts of human operators by providing corrective actions which are based entirely and strictly on a set of objective rules. Referring to “Calculation 1” as an example, expert human operator A may judge that a Diversion Tank Level of 85% is still within the safety limit for wastewater diversion without knowing that expert human operator B has previously encountered a critical failure by diverting wastewater into the Diversion Tank when the level is at 85%.

Moreover, rule-based or logic-based recommendation database greatly facilitates the addition of new knowledge. Referring to “Calculation 1”, the addition of a new root cause into the recommendation database that involves a corrective action which similarly requires at least the diversion of wastewater to the diversion tank will automatically be flagged to follow the same rules as laid out in “Calculation 1”.

Further, it may be observed that determining the appropriate corrective action from a list may involve the consideration of many different factors and rules. Rule-based or logic-based recommendation database 40 may eradicate conflicting instructions and maintains consistency in decision making. In various embodiments, it may also ensure that the addition of new corrective actions does not conflict with existing ones by making sure that the triggering conditions are mutually exclusive, i.e. the Boolean logic returns only one unique result. Another advantage of the rule-based recommendation database which encodes the knowledge of domain experts is to enable non-technical people to easily experiment with different rules or safety limits that deviate from those imparted from the domain experts either in a simulated or actual context.

Upon determining the corrective instructions based on the above methods, the Troubleshooting module 14 may be operable to summarize all the quantitative and qualitative corrective instructions as “Recommendations” for display to the user in order of weightage.

Although the Troubleshooting module 14 may provide the appropriate corrective instructions to restore the root causes corresponding to the abnormal process parameters, it does not provide information about the possible impact of the undertaking the corrective instructions.

In some embodiments, the troubleshooting module 14 may be independent from the Prediction Module 12, and operable to receive the dataset in the form of one or more electronic files. The dataset may be obtained directly from the historical database 30 without any form of prediction.

To obtain more information about the possible impact of the undertaking the corrective instructions as generated by the troubleshooting module, the user may activate the Prognosis module 16.

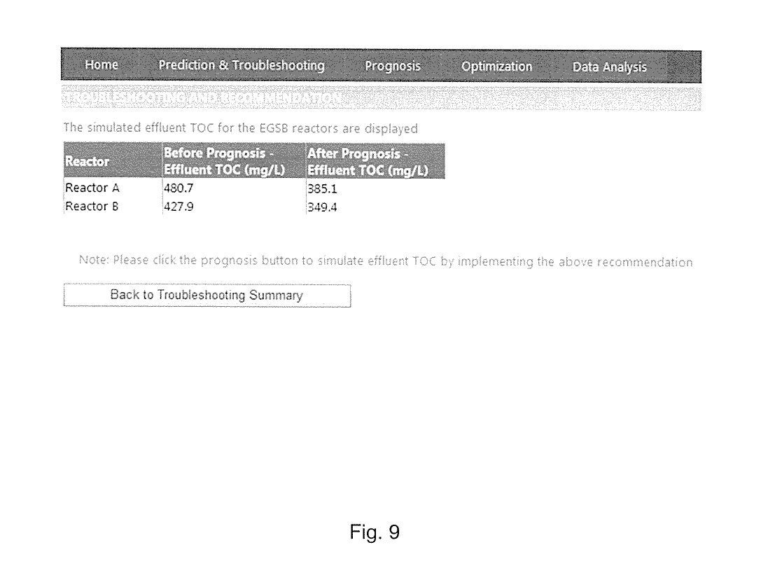

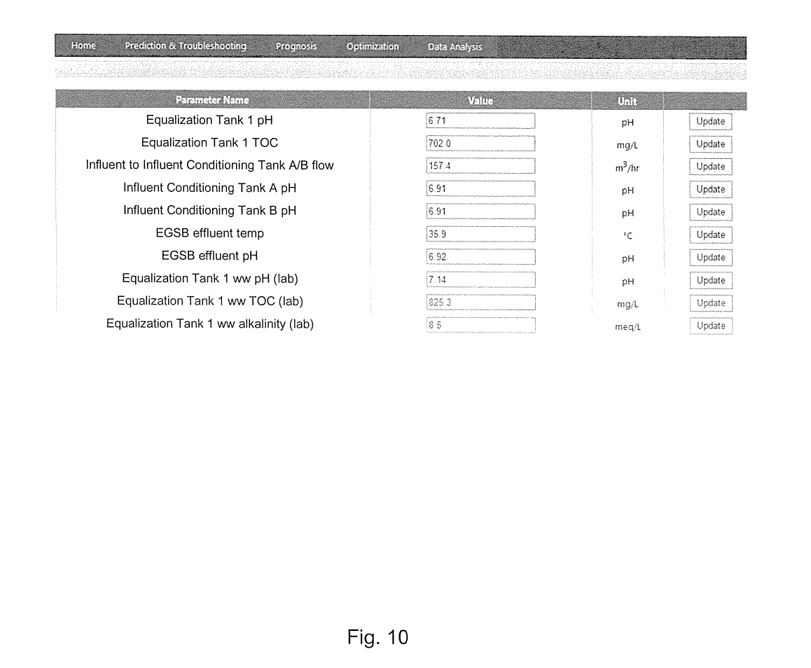

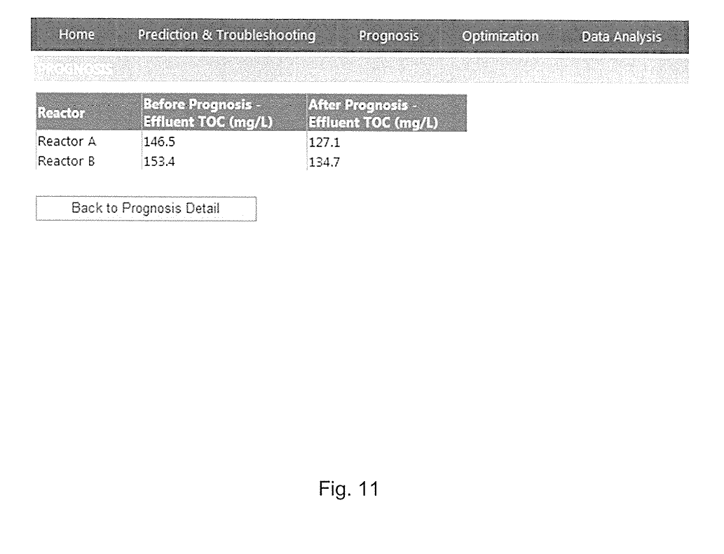

In various embodiments, the Prognosis Module 16 has two modes—integrated prognosis and independent prognosis modes. Integrated prognosis mode is a supplement function to the troubleshooting module 14 that is activated in step 216 to project or simulate the impact of the corrective instructions on the effluent parameter before implementing the recommended corrective instructions in step 214. On the other hand, the independent prognosis mode allows the user to manually change certain input parameter/parameters and project the treatment performance even without activating troubleshooting module 14. FIG. 9 shows the simulated effect of adopting the corrective actions generated by the troubleshooting module 14 in the integrated prognosis mode. Referring to FIG. 10, the Prognosis module 16 provides a graphical user interface comprising a list of the controllable process parameters and their current values. In independent prognosis as illustrated in FIG. 10, the list may include process parameter(s) which are predetermined. The graphical user interface further allows the user to manually adjust the values individually or collectively for the purpose of projecting the effluent parameter (TOC). The projected effluent TOC is then displayed as shown in FIG. 11. In various embodiments, the simulation or projection may be performed using heuristic or statistical method similar to the Performance Prediction Module. In integrated prognosis (not shown), the list may include process parameters which have been identified as controllable root causes.

While the Prognosis Module 16 allows the user to manually project or simulate the impact of the unique combination of corrective instructions on the effluent parameter, it typically does not provide the solution to restore the effluent parameter to an optimum level or range. Although it may be possible to determine the optimum solution via the Prognosis Module 16 using a manual trial-and-error or other methods such as Monte Carlo, such methods are typically time consuming and involves guess-work. An alternative and advanced solution provided in this invention is to activate the Optimization module 18. The objective of the activating the Optimization module 18 as depicted in step 220 is to return the effluent parameter (TOC) to an optimum level and provide the user with:

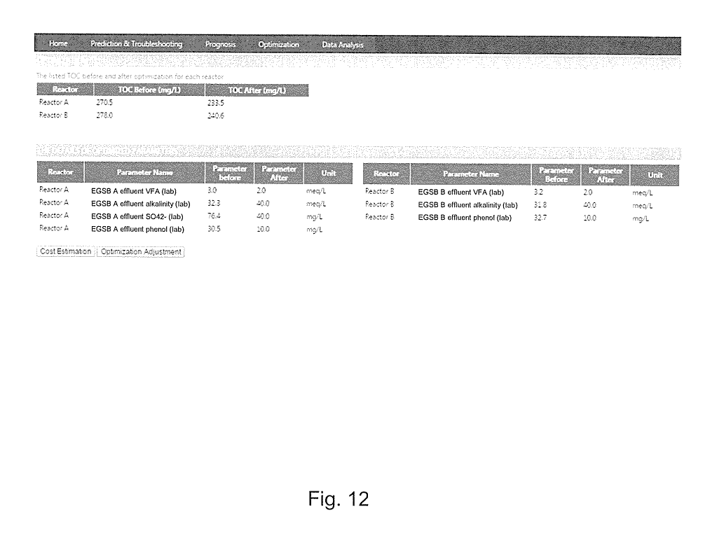

1) a process target for each corresponding process parameter (FIG. 12), and

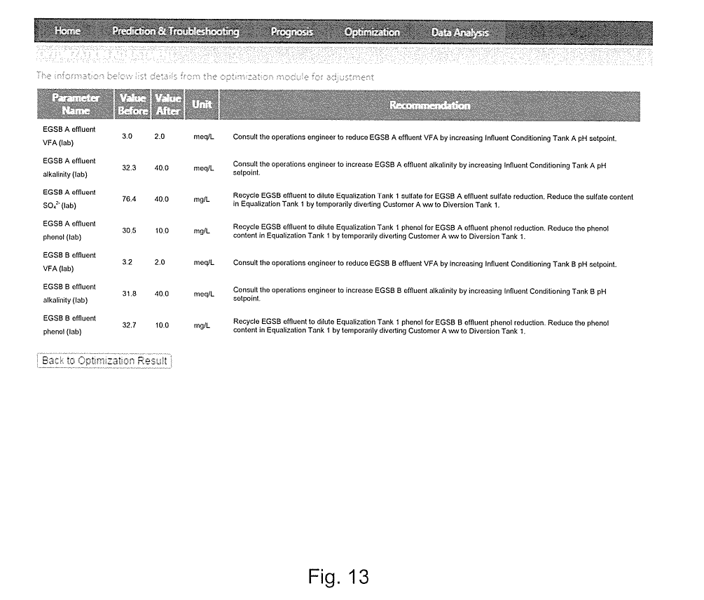

2) corrective instructions on how to achieve the optimum result (FIG. 13).

It is to be appreciated that the Optimization module 18 can be activated even when the effluent parameter is within its normal range, and not necessarily has to be activated after troubleshooting or prognosis.

Referring to FIGS. 12 and 13, a total of seven (7) process parameters are identified with their current values listed under the “Value Before” field. The “Value After” field provides the value for each process parameters/variables to optimize the effluent parameter and the “Recommendation” field provides the corrective instruction required to attain an optimum scenario. It is to be appreciated that the identified process parameters may not need to be controllable as the Optimization module is capable of providing corrective instructions to the user to indirectly adjust other parameters such as pH, influent water alkalinity, reactor performance etc. In various embodiments, the process parameters are selected with the principle of identifying process parameters that can be used as indicators when optimizing the process to reduce the EGSB's TOC. These parameters have been selected based on operator and process knowledge. For example, reducing the EGSB effluent Volatile Fatty Acids (VFA) helps in minimizing the eventual TOC of the EGSB effluent stream.

In various embodiments, it is to be appreciated that the optimization may be performed by optimizing different objective functions. It is to be appreciated that the step of optimization typically includes either maximizing or minimizing one or more objective functions, depending on the nature of problem. Considering different objectives (treatment results and operational costs) and different environmental conditions in which the plant has to operate (wastewater quality, flow and temperature fluctuations) significantly increases the complexity of the problem. For example, the optimal operation of a wastewater treatment plant involves for example selecting appropriate sludge circulation flows and chemical dosing rates such that they optimize the behaviour of the plant, according to some pre-defined criteria, in given conditions. According to the developed PLS model,

y=x₁b₁+x₂b₂+ . . . +xₙbₙ (2)

the coefficients are obtained. Each process parameter has a specific operational range. Then the optimization objective function as described below is developed to optimize the treatment efficiency, by a linear optimization to get optimized process parameters and the optimized effluent TOC, where lb and ub are the lower and upper boundaries, respectively.

minimize(y)=minimize(x₁b₁+x₂b₂+ . . . +xₙbₙ) (3)

subject to: lb₁≤x₁≤ub₁, . . . ,lbₙ≤xₙ≤ubₙ

In various embodiments, the optimization process may be performed with respect to the objective function relating to the overall operational or operating cost. The direct material cost for operating the EGSB increases after the implementation of corrective instructions. However, the downstream operating cost may be reduced due to a lower effluent TOC from the EGSB as a consequence of implementing the corrective actions. As such, the optimum overall operating cost (including both the EGSB and further downstream processes) is a delicate trade-off between the increase in direct material cost for implementing the corrective actions and the reduction in downstream operating cost due to lower TOC.

In various embodiments, the direct material cost is calculated by taking into account the cost of chemical addition (Hydrochloric acid—HCl, sodium hydroxide (caustic)—NaOH—adjust pH, H₃PO₄, ammonia as nutrient) and also power cost for running electrical equipment. In general, operating cost can be reduced via two means of the modules:

1) Avoiding high cost that can arise due to serious process upsets—“avoid process cost”—Troubleshooting module

2) Maximizing TOC removal at the EGSB process and therefore reduce TOC load to the downstream aerobic process, where sludge generation and air requirement is significant.

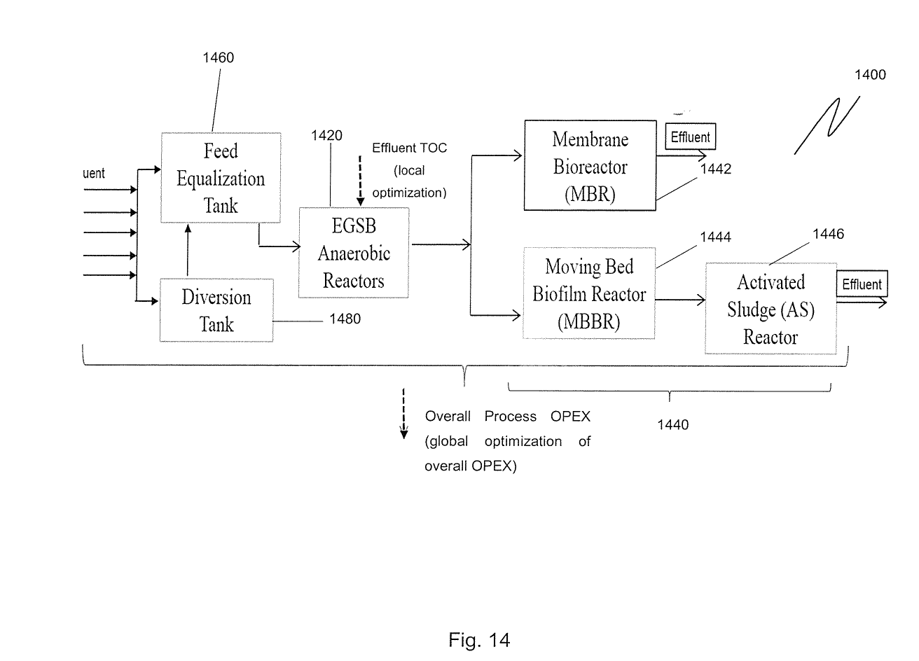

In some embodiments, the system 10 or part of the system 10 such as prediction module 12, troubleshooting module 14 and optimization module 18 may be applied to a wastewater treatment system 1400 comprising an anaerobic sub-system 1420 and an aerobic sub-system 1440. The anaerobic sub-system 1420 and the aerobic sub-system 1440 are arranged such that the anaerobic sub-system 1420 is upstream with respect to the downstream aerobic sub-system 1440. As illustrated in FIG. 14, the system 1400 comprises a feed equalization tank 1460 and a diversion tank 1480 arranged to receive influent (wastewater). Influent from the diversion tank 1480 may be fed to the feed equalization tank 1460 at an adjustable or controllable diversion flow rate (amongst other functions).

The anaerobic sub-system 1420 may comprise at least one EGSB anaerobic reactor arranged to receive regulated influent from the feed equalization tank 1460. Such EGSB anaerobic reactor may be similar to the EGSB anaerobic reactor 102. After an anaerobic treatment process, the treated wastewater is sent downstream to the aerobic sub-system 1440. As illustrated, the aerobic sub-system 1440 comprises a membrane bioreactor (MBR) 1442, a moving bed biofilm reactor (MBBR) 1444, and an activated sludge (AS) reactor 1446. Treated wastewater output effluent from the MBR or AS reactor may be sent to subsequent process systems or sub-systems.

The process parameters or variables of the at least one EGSB reactor (such as EGSB reactor 102) may be optimized to minimize effluent TOC (effluent from the EGSB reactor) considering only the upstream anaerobic sub-system as described in earlier embodiments.

In optimizing system 1400, the overall or global optimization of overall OPEX is considered. This may also be achieved by minimizing the effluent TOC concentration in the upstream EGSB anaerobic sub-system 1420, and/or maximizing the effluent TOC concentration in the downstream aerobic sub-system 1440. In some embodiments, any optimized results from the upstream anaerobic sub-system 1420 may be used as an advisory method to advice operators any possible adjustments that allows the discharged effluent TOC from the EGSB reactor to meet any regulatory requirement(s) in the aerobic downstream 1440. In the maximization of the downstream aerobic effluent TOC, an operator will be advised to treat the effluent wastewater such that the effluent wastewater would just meet the regulatory requirements. For example, if the regulatory requirement is for treated downstream wastewater to have effluent TOC of less than 33 mg/L and if the operator is currently treating the downstream wastewater to 25 mg/L, there is room for the TOC of the discharge effluent to be increased slightly (e.g. 30 mg/L). There is no need to “over-treat” the wastewater at the downstream aerobic processes. Lower treatment requirements at the downstream aerobic process would result OPEX reduction due to lesser sludge generation, lower aeration power consumption.

In the embodiment of FIG. 14, the optimization may be performed without the need for the troubleshooting module 14 and prognosis module 16. In comparison with the earlier described embodiments, the optimization module 18 takes into account optimization of the downstream aerobic sub-system 1440 instead of merely the anaerobic sub-system 1420.

In another embodiment, the optimization module 18 may work in conjunction with the troubleshooting module 14 to identify root cause(s) so as to restore a wastewater treatment system or sub-system to normal, and concurrently/simultaneously provide an optimal solution with respect to a predetermined objective function during the restoration process. The optimization module 18 is configured to create permutations and combinations of corrective actions associated with each identified root cause. Each permutation or combination maybe derived from one or more corrective action.

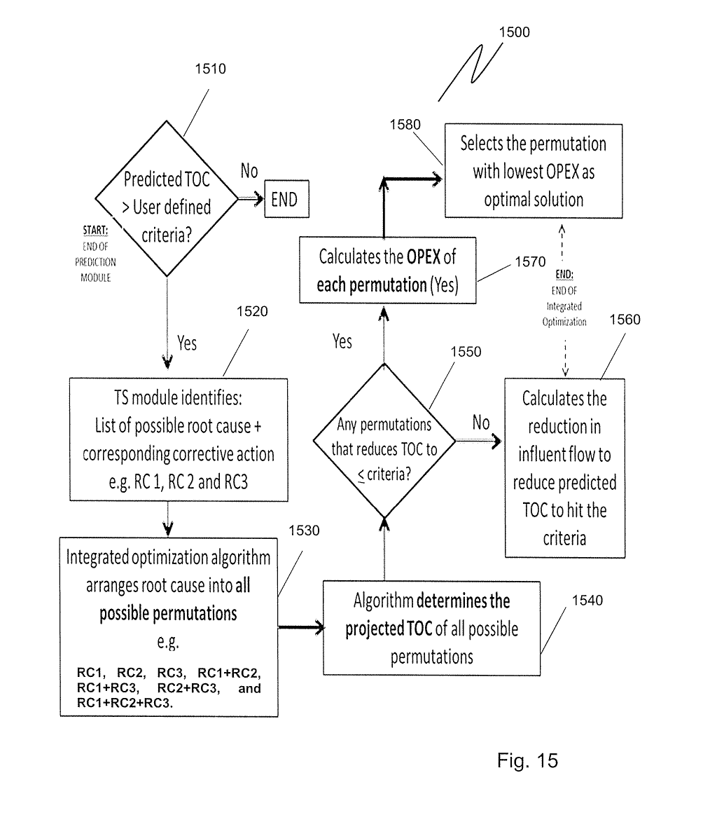

The optimization module 18 may be applied to the EGSB reactor 102 or any other wastewater treatment or sub-systems. As an example, FIG. 15 illustrates a method 1500 of optimizing the predetermined objective function OPEX based on an output obtained from the prediction module 12.

At step 1510, the predicted effluent such as Total Organic Carbon (TOC) is compared with a user defined criterion/criteria, which may be a maximum value of TOC allowable. If the predicted TOC is more than the user defined criteria, thereby signifying at least one abnormality, the list of possible root causes and corresponding controllable process variable(s) are identified using the troubleshooting module 14 (step 1520). The corresponding corrective actions (via troubleshooting module 14) are generated.

For purpose of illustration, the corresponding corrective actions are expressed as RC1, RC2, and RC3. These may correspond to adjustments in temperature, influent concentration, or recirculation flow. The optimization module 16 then computes all possible permutations of corrective actions (including combination of 2 or more possible corrective actions), i.e. RC1; RC2; RC3; RC1+RC2, RC1+RC3; RC2+RC3; and RC1+RC2+RC3. (step 1530). Based on the three examples, seven possible combinations are computed in accordance with the mathematical expression:

$${\sum }_{r = 1}^{n} C ( n , r )$$

Where n denotes the total number of corrective actions identified.

The corresponding projected TOC based on each of the seven corrective action(s) is then calculated or computed (step 1540). Each of the seven corresponding TOC is then compared against the user defined criterion/criteria, and all permutations that reduce TOC to less than the criteria are identified (step 1550). If there is no available solution that reduces TOC to less than or equal the user defined criteria, a default action, such as a reduction in influent flow to reduce predicted TOC to hit the criteria, will be calculated or determined (step 1560). This is in effect the ‘next best’ solution.

If there is at least one available solution to reduce the TOC to less than the criteria, then the OPEX of each available solution is computed (step 1570). The solution with the lowest OPEX is selected as the optimal solution (1580).

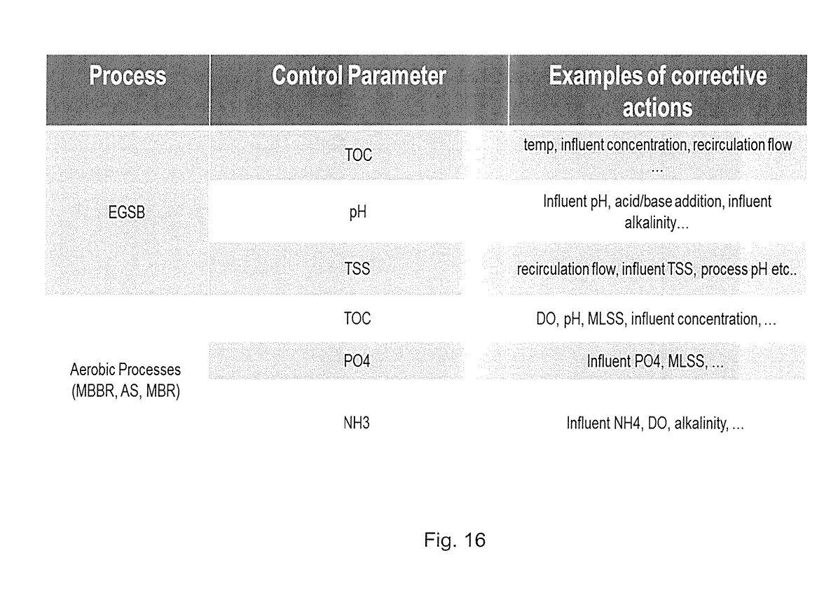

FIG. 16 shows exemplary applications of the method to optimize the corrective actions in accordance with some embodiments of the invention. In particular, the parameters to be optimized at the anaerobic sub-system 1420 may comprise TOC, pH, and/or total suspended solids (TSS). The parameters that may be optimized for aerobic sub-system comprises minimization of MBBR, AS, and MBR. These may include corrective actions associated with effluent TOC, effluent phosphate PO₄ and effluent Ammonia NH₃.

For each of the control parameter to be optimized, examples of corrective actions are illustrated in FIG. 16. For example, when optimizing the control parameter effluent pH for the EGSB sub-system, the corrective actions may include adjustments to influent pH, acid/base addition, and/or influent alkalinity.

In summary, the purpose of identifying one or more root causes is to restore the system 100, 1400 to a status as defined by the user defined criteria.

The integrated optimization process selects the lowest cost combination of corrective actions to meet this criteria. If no combination of the identified root cause can result in the system 100, 1400 meeting the user criteria, the “next best” solution would be for the user to reduce the load on the treatment system, so that the discharge (effluent) parameter can meet this defined criteria.

In some embodiments, optimization of the wastewater treatment system 1400 may be modelled as minimization of a parameter for the upstream anaerobic sub-system 1420 and the maximization of the same parameter for the downstream aerobic sub-system 1440 to achieve overall OPEX reduction. In other embodiments, the optimization of the wastewater treatment system 1400 may be modelled as maximization of a parameter for the upstream anaerobic sub-system 1420 and the minimization of the same parameter for the downstream aerobic sub-system 1440 to achieve overall OPEX reduction.

It should be further appreciated by the person skilled in the art that variations and combinations of features described above, not being alternatives or substitutes, may be combined to form yet further embodiments falling within the intended scope of the invention. In particular,
Although in the described embodiment the invention is discussed in the context of EGSB reactor and a general anaerobic and aerobic system, it is appreciated that the present invention may be applicable to other arrangements or configurations of WWTPs. In particular, it is to be appreciated that the process variables and process parameters as described may be applicable to other aerobic and/or anaerobic wastewater treatment process and systems, and data associated with these process variables and process parameters collected in the form of historical database(s) 30 and recommendation database(s) 40 specific to the type of WWTP.
Although as illustrated in FIG. 2 and FIG. 3, the Prognosis module 16 and Optimization module 18 are implemented following the troubleshooting module 14, it is to be appreciated that the two modules may also be run independently from the troubleshooting module 14.
In various embodiments, the optimization module 18 may be directly integrated with the Troubleshooting module 14 instead of a two-step process implementation as exemplified in FIG. 2.
Although the recommendation database 40 is rule-based or logic-based, it is to be appreciated that other forms of cognitive models which are capable of decision making may also be applicable.
Although the recommendation database 40 is based on classical Boolean logic whereby the outcome is binary (true or false), it is to be appreciated that other forms of logical model such as Fuzzy logic may also be used in the invention. In some embodiments, hybrid between two or more logical models may be adopted.

1. A system for wastewater treatment process control comprising
a plurality of measuring means arranged to obtain a dataset, the dataset comprises a plurality of process variables related to a parameter of the wastewater treatment process;
a prediction module arranged to receive the dataset and predict the parameter of wastewater treatment process utilizing a mathematical model, the mathematical model arranged to obtain the dataset as input and provide a predicted parameter as an output;
a troubleshooting module arranged to compare the predicted parameter with a predetermined criterion;
wherein if the predicted parameter does not satisfy the predetermined criterion; the troubleshooting module is operable to identify at least one process variable from the plurality of process variables which causes the predicted parameter not to satisfy the predetermined criterion, and determine whether the identified at least one process variable from the plurality of process variables is controllable.

2. The system according to claim 1, wherein the parameter of the wastewater treatment process is an effluent parameter of the wastewater treatment process.

3. The system according to claim 1 or 2, wherein the mathematical model comprises a moving-window partial least squares regression algorithm.

4. The system according to any one of the preceding claims, wherein the identification of the at least one process variable which causes the predicted parameter not to satisfy the predetermined criterion is based on a Hotelling T² or Q/SPE statistic.

5. The system according to any one of the preceding claims, wherein the predetermined criterion is in the form of a maximum value allowable, a range of acceptable or allowable values, or a minimum value allowable.

6. The system according to claim 1, wherein if the at least one process variable is controllable, the troubleshooting module obtains the median value of the at least one controllable process variable and determines if the at least one controllable process variable is a root cause.

7. The system according to claim 6, wherein the determination of whether the at least one controllable process variable is a root cause includes comparing the median value of the at least one controllable process variable against a range of values the at least controllable process variable operates under normal condition.

8. The system according to claim 7, wherein if the controllable process variable falls outside the range of values, the controllable process variable is classified as a root cause.

9. The system according to claim 8, wherein the root cause is further classified as either a qualitative or a quantitative root cause.

10. The system according to claim 9, wherein if the root cause is a quantitative root cause, further calculations are provided to calculate an adjustment to the at least one process variable.

11. The system according to claim 9, wherein the troubleshooting module is operable to access a database to retrieve at least one corrective instruction to adjust the controllable process variable based on a set of pre-defined rules.

12. The system according to any one of the preceding claims, further comprising a prognosis module operable to simulate the impact of the adjustment of the at least one controllable process variable on the parameter of the wastewater treatment process.

13. The system according to any one of claims 1 to 11, further comprising a prognosis module operable to simulate the impact of adjustments of at least one process variable on the parameter of the wastewater treatment process.

14. The system according to any one of the preceding claims, further comprises an optimization module to optimize the plurality of process variables and parameter of wastewater treatment process with respect to at least one objective function.

15. A method for wastewater treatment process control comprising the steps of:
obtaining from a plurality of measuring means a dataset, the dataset comprises a plurality of process variables related to a parameter of the wastewater treatment process;
receiving the dataset at a prediction module and predicting the parameter of wastewater treatment process based on a mathematical model;
comparing the predicted parameter with a predetermined criterion;
wherein if the predicted parameter does not satisfy the predetermined criterion; the troubleshooting module is operable to identify at least one process variable from the plurality of process variables which causes the predicted parameter not to satisfy the predetermined criterion and determine whether the identified at least one process variable from the plurality of process variables is controllable.

16. A troubleshooting module for use in wastewater treatment process control, comprising at least one processor in data communication with a plurality of measuring means to receive a dataset, the dataset comprises a plurality of process variables related to a parameter of the wastewater treatment process, and a value of the parameter of the wastewater treatment process; and thereafter compare the value of the parameter with a predetermined criterion;
wherein if the parameter does not satisfy the predetermined criterion; the troubleshooting module is operable to identify at least one process variable from the plurality of process variables which causes the predicted parameter not to satisfy the predetermined criterion and determine whether the identified at least one process variable from the plurality of process variables is controllable.

17. The troubleshooting module according to claim 16, wherein the identification of the at least one process variable which causes the predicted parameter not to satisfy the predetermined criterion is based on a Hotelling T² or Q/SPE statistic.

18. The troubleshooting module according to claim 16 or 17, wherein the predetermined criterion is in the form of a maximum value allowable, a range of acceptable or allowable values, or a minimum value allowable.

19. The troubleshooting module according to claim 16, wherein if the at least one process variable is controllable, the median value of the at least one controllable process variable is obtained and the troubleshooting module is operable to determine if the at least one controllable process variable is a root cause.

20. The troubleshooting module according to claim 19, wherein the determination of whether the at least one controllable process variable is a root cause includes comparing the median value of the at least one controllable process variable against a normal range.

21. The troubleshooting module according to claim 20, wherein if the controllable process variable falls outside the normal range, the controllable process variable is classified as a root cause.

22. The troubleshooting module according to claim 21, wherein the root cause is further classified as either a qualitative or a quantitative root cause.

23. The troubleshooting module according to claim 22, wherein if the root cause is a quantitative root cause, an adjustment to the process variable is calculated.

24. The troubleshooting module according to claim 23, wherein the troubleshooting module is operable to access a database to retrieve at least one corrective instruction to adjust the controllable process variable based on pre-defined rules.

25. The troubleshooting module according to any one of claims 16 to 24, further comprising a prognosis module operable to simulate the impact of the adjustment of the controllable process variable on the parameter.

26. The troubleshooting module according to claim 16, further comprising a prognosis module operable to simulate the impact of adjustments of at least one process variable on the parameter.

27. The troubleshooting module according to claim 23, further comprises a list of a plurality of quantitative and/or qualitative root causes and corresponding correction actions, wherein each corresponding adjustment is a corrective action.

28. The troubleshooting module according to claim 27, further comprises an optimization module arranged to permute the corrective actions in various combinations in accordance with the formula
$${\sum }_{r = 1}^{n} C ( n , r )$$
where n denotes the total number of corrective actions identified.

29. The troubleshooting module according to claim 28, wherein a simulated parameter corresponding to each corrective action is determined and each simulated parameter is compared with the predetermined criterion.

30. The troubleshooting module according to claim 29, wherein if the simulated parameter satisfies the predetermined criterion, an objective function value is calculated, and where there comprises a plurality of objective function values, the lowest or highest objective function value is selected as an optimal solution.

31. The troubleshooting module according to claim 29 or 30, wherein if no simulated parameter is found to satisfy the predetermined criterion, a next best alternative based on adjusting a predetermined process variable is selected as the optimal solution.

32. An optimization module for use in wastewater treatment system, the wastewater treatment system comprises at least one anaerobic sub-system and at least one aerobic sub-system, the optimization module comprises at least one processor arranged in data communication with a plurality of measuring means to receive a dataset, the dataset comprises a plurality of process variables related to a parameter of the wastewater treatment process;
wherein the at least one processor is operable to optimize the parameter and the plurality of process variables with respect to an objective function, and wherein the objective function comprises minimization of an overall operating expense of the wastewater treatment system.

33. The optimization module of claim 32, wherein the minimization of the overall operating expense comprises minimizing the parameter in the anaerobic sub-system.

34. The optimization module of claim 32 or 33, wherein the minimization of the overall operating expense comprises maximizing the parameter in the aerobic sub-system.

35. The optimization module of claim 33 or 34, wherein the parameter is the effluent TOC concentration of the anaerobic or aerobic sub-systems.
